(19) United States
(12) Patent Application Publication
(10) Pub. No.: US 2024/0343776 A1
(43) Pub. Date: Oct. 17, 2024

(54) PROCESS FOR PURIFICATION OF FUSION PROTEIN

(71) Applicant: Kashiv BioSciences, LLC, Piscataway, NJ (US)

(72) Inventors: Om Narayan, Ahmedabad (IN); Kishor Galani, Surat (IN); Rupen Bhavsar, Ahmedabad (IN); Roshan Ganeshlal Upadhyay, Ahmedabad (IN)

(21) Appl. No.: 18/620,255

(22) Filed: Mar. 28, 2024

Related U.S. Application Data
(63) Continuation of application No. PCT/IB22/59539, filed on Sep. 28, 2022.

(30) Foreign Application Priority Data
Sep. 28, 2021 (IN) 202121043967

(51) Int. Cl. C07K 14/705; C07K 1/18

(52) U.S. Cl. CPC C07K 14/70521; C07K 1/18; C07K 2319/30

(57) ABSTRACT

The present invention is directed to the use of anion exchange chromatography to produce a CTLA4-Ig fusion protein with improve glycan.

### CROSS-REFERENCE TO RELATED APPLICATIONS

This application is a Continuation of International Application No. PCT/IB2022/059239, filed on Sep. 28, 2022, which claims the benefit of and priority to Indian Patent Application No. 202121043967, filed on Sep. 28, 2021, each of which is incorporated by reference herein in its entirety.

### FIELD OF THE INVENTION

The present invention is directed to the use of anion exchange chromatography to produce a CTLA4-Ig fusion protein with improve glycan.

### BACKGROUND OF THE INVENTION

Fusion proteins are complex in nature as made of fusion of receptor (natural or modified) and immunoglobulin constant region (Fc including with or without hinge region or modified Fc). As per the complexity of the fusion proteins and its challenging purification process gives a motivation to minimize the impurities from fusion protein that proportionally affect the stability and functional efficacy. The recent advances in mammalian cell culture processes have significantly increased product titers as well as process and product-related impurities. Aggregation, charge variants, high molecular weight (HMW), low molecular weight (LMW) like impurities with the fusion protein has been a major problem that has been associated with a change in protein structure and being a hurdle in various upstream and downstream purification processes. The Fc-fusion proteins have elevated levels of aggregates, high molecular weight species (HMWs; up to 20%) and low molecular weight species (LMWs; up to 20%) within the product species. Glycosylation of proteins and the subsequent processing of the added carbohydrates can affect protein folding and structure, protein stability, including protein half-life, and functional properties of a protein. Desired glycosylation can be obtained through adequate clone, upstream and/or downstream process. The present invention provides the improvement in glycan in fusion protein by using only one anion exchange chromatography. There is a present need for methods of producing and purifying a fusion protein of interest in sufficiently pure form to be suitable for pharmaceutical use.

### SUMMARY OF THE INVENTION

The present invention identified the use of anion exchange chromatography (AEX) to improve the undesired glycan from fusion protein.

In certain embodiment, the AEX is strong anion exchange chromatography.

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the washing is performed;
c. eluting the CTLA4-Ig fusion protein mixture from anion exchange column;
wherein the CTLA4-Ig fusion protein has more than 50% improvement in high mannose.

In an embodiment the CTLA4-Ig fusion protein has more than about 50%, about 51%, about 52%, about 53%, about 54%, about 55%, about 56%, about 57%, about 58%, about 59%, about 60% improvement in high mannose.

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the washing is performed;
c. Eluting the CTLA4-Ig fusion protein mixture from anion exchange column;
wherein the CTLA4-Ig fusion protein has low or reduce high mannose by at least 50%.

In such embodiment, the high mannose is reduced about 50%, about 55%, about 60%, about 65%, about 70%, about 75%, about 80%.

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the washing is performed;
c. Eluting the CTLA4-Ig fusion protein mixture from anion exchange column;
wherein the CTLA4-Ig fusion protein has reduce afucosylation by at least 20%.

In such embodiment, the afucosylation is reduced from about 20% to about 30%.

In certain embodiment, the afucosylation is reduced about 20%, about 21%, about 22%, about 23%, about 24%, about 25%, about 26%, about 27%, about 28%, about 29%, and about 30%.

In an embodiment, the purification of CTLA4-Ig fusion protein by performing anion exchange chromatography, optionally further comprises one or more chromatography step can be employed before or after anion exchange chromatography.

In certain embodiment the Affinity chromatography is performed before the anion exchange chromatography.

### DETAILED DESCRIPTION OF THE INVENTION

The present invention identified the use of anion exchange chromatography (AEX) to improve the undesired glycan from fusion protein.

In certain embodiment, the AEX is strong anion exchange chromatography.

As used herein the term “column” or “resin” or “chromatographic resin or chromatographic column” are interchangeable.

The term “comprises” or “comprising” is used in the present description, it does not exclude other elements or steps. For purpose of the present invention, the term “consisting of” is considered to be an optional embodiment, of the term “comprising of”. If hereinafter a group is defined to comprise at least a certain number of embodiments, this is also to be understood to disclose a group which optionally consists only of these embodiments. As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “Protein A affinity chromatography” use for the separation or purification of substances and/or particles using protein A, where the protein A is generally immobilized on a solid phase. Protein A is a 40-60 kD cell wall protein originally found in Staphylococcus aureus. The binding of fusion protein to protein A resin is highly specific. Protein A affinity chromatography columns for use in protein A affinity chromatography herein include, but are not limited to, Protein A immobilized on a polyvinyl ether solid phase, e.g., the Eshmuno® columns (Merck, Darmstadt, Germany), Protein A immobilized on a pore glass matrix, e.g., the ProSep® columns (Merck, Darmstadt, Germany) Protein A immobilized on an agarose solid phase, for instance the MABSELECT™ SuRe™ columns (GE Healthcare, Uppsala, Sweden).

The term “MabSelect SuRe LX” used herein is a protein A affinity resin with high dynamic binding capacity at extended residence times. The ligand is alkali-stabilized protein A-derived (E. coli) shows alkali tolerance, high capacity and low ligand leakage in combination with the rigid base matrix.

### Anion Exchange Chromatography (AEX)

The term “anion exchange chromatography” or “anion exchange column” or “AEX” used herein is a form of ion exchange chromatography (IEX), which is used to separate molecules based on their net surface charge. Anion exchange chromatography, more specifically, uses a positively charged ion exchange resin with an affinity for molecules having net negative surface charges. Anion exchange chromatography is used both for preparative and analytical purposes and can separate a large range of molecules, from amino acids and nucleotides to large proteins. Here, we focus on the preparative anion exchange chromatography of proteins.

As used herein the term “bind and elute mode” or “B/E” refers to purification process wherein the fusion protein of interest binds to chromatography resin. At least 90% fusion protein of interest bind to chromatographic resin. At least 60% or 70% or 80% fusion protein of interest binds to chromatographic resin. However, process and product related impurities does not bind the chromatographic resin. At least 50% process and product related impurities does not bind to chromatographic resin. At least 60% or 70% or 80% process and product related impurities does not bind to chromatographic resin.

In an embodiment the anion exchange is strong anion exchange. The term “POROS XQ” used herein is a Thermo Scientific POROS XQ Strong Anion Exchange Resin are designed for charge based chromatographic separations of biomolecules including recombinant proteins, or fusion proteins. POROS XQ has high and consistent protein capacity across a broad range of salt concentrations. The resin has quaternary amine groups.

### Buffer

The term “buffer” or “suitable buffer” refers to a solution that can resist pH change upon the addition of an acidic or basic components. It is able to neutralize small amounts of added acid or base, thus maintaining the pH of the solution relatively stable. This is important for processes and/or reactions which require specific and stable pH ranges. Buffer solutions have a working pH range and capacity which dictate how much acid/base can be neutralized before pH changes, and the amount by which it will change.

The term used herein “Buffer B” or “Elution buffer B” in anion exchange chromatography are interchangeable. For a non-limiting example, it refers to the buffer solution of 20 mM sodium phosphate, 300 mM sodium chloride at pH 7.2±0.2, conductivity 30.0±3.0 mS/cm.

The term “clarified harvest cell culture fluid” or “HCCF” or “harvest cell culture fluid” used herein are interchangeable refers to the protein mixture obtained from mammalian cell culture containing protein of interest along with other impurities.

The term “N-Glycan” used herein refers to the method that determines the total individual sugar molecule present in the fusion protein.

The term “High Mannose” used herein refers to the sugar mannose present in the fusion protein molecule.

The term “Afucosylation” used herein refers to the protein molecule do not have any fucose sugar units.

The term “Galactosylation” used herein refers to the protein molecule containing or adding sugar galactose on the N-linked site of the protein molecule.

The term “undesired glycan” used herein refers to the N-glycan of the fusion protein that is beyond the acceptable range. In an embodiment, the acceptable N-glycan ranges includes high mannose, afucosylation, galactosylation. The N-glycan profile of fusion protein should be in the acceptable range as mentioned above to comply the regulatory requirements.

The term “substantially pure fusion protein” used herein includes a fusion protein that is substantially free of impurity selected from product or process related impurities. The fusion protein is free of acidic variants, basic variants, low molecular weights and high molecular weights, substantially pure fusion protein has purity less than about 99% or less than about 98% or less than about 97% or less than about 95% or less than about 92% or less than about 90% or less than about 88% or less than about 85% or less than about 82% less than about 80% or less than about 75% or less than about 70% or less than about 65% or less than about 60% or less than 50%. The term “Residence time” refers to the amount of time a compound spends on the column after it has been injected. If a sample containing several compounds, each compound in the sample will spend a different amount of time on the column according to its chemical composition i.e., each will have a different retention time. Retention times are usually quoted in units of seconds or minutes.

The terms “CTLA4-Ig” or “CTLA4-Ig molecule” or “CTLA4-Fc molecule” or “CTLA4-Ig fusion protein” are used interchangeably and refer to a protein molecule that comprises at least a polypeptide having a CTLA4 extracellular domain or portion thereof and an immunoglobulin constant region or portion thereof. The extracellular domain and the immunoglobulin constant region can be wild-type, or mutant or modified, and mammalian, including human or mouse. The polypeptide can further comprise additional protein domains. A CTLA4-Ig molecule can also refer to multimer forms of the polypeptide, such as dimers, tetramers, and hexamers. A CTLA4-Ig molecule is also capable of binding to CD80 and/or CD86. In an embodiment, the CTLA4-Ig is Abatacept.

The term “Drug Substance” or “DS” refers to an active ingredient intended to furnish pharmacological activity. The DS also refers to final protein mixture after all purification steps and substantially free from impurities.

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the wash is performed;
c. eluting the CTLA4-Ig fusion protein mixture from anion exchange column;
   wherein the CTLA4-Ig fusion protein has more than 50% improved of high mannose.

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the wash is performed;
c. eluting the CTLA4-Ig fusion protein mixture from anion exchange column;
   wherein the CTLA4-Ig fusion protein has high mannose low or reduce by at least 50%.

In such embodiment, the high mannose is reduced about 50%, about 55%, about 60%, about 65%, about 70%, about 75%, about 80%

In an embodiment, a process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the wash is performed;
c. eluting the CTLA4-Ig fusion protein mixture from anion exchange column;
   wherein the CTLA4-Ig fusion protein has afucosylation reduce by at least 20%.

In such embodiment, the afucosylation is reduced from about 20% to about 30%.

In certain embodiment, the afucosylation is reduced about 20%, about 21%, about 22%, about 23%, about 24%, about 25%, about 26%, about 27%, about 28%, about 29%, and about 30%.

In an embodiment, the invention is related to the purification of CTLA4-Ig fusion protein mixture comprising:
a) loading the fusion protein mixture onto first chromatography column;
b) optionally performing the washing;
c) eluting the fusion protein mixture from said first chromatography column;
d) optionally performing the filtration;
e) loading the fusion protein mixture obtained from step (C) or (D) onto anion exchange chromatography column;
f) optionally performing the washing;
g) eluting the fusion protein mixture from said anion exchange chromatography column; wherein the first chromatography column is selected from Protein-A column, Hydrophobic interaction chromatography, cation exchange chromatography.

In an embodiment, the invention is related to the purification of CTLA4-Ig fusion protein mixture comprising:
a) loading the fusion protein mixture onto first chromatography column;
b) optionally performing the washing;
c) eluting the fusion protein mixture from said first chromatography column;
d) optionally performing the filtration;
e) loading the fusion protein mixture obtained from step (C) or (D) onto anion exchange chromatography column;
f) optionally performing the washing;
g) eluting the fusion protein mixture from said anion exchange chromatography column;
h) loading the fusion protein mixture onto third chromatography column;
i) optionally performing the washing;
j) eluting the fusion protein mixture from third chromatography column
wherein the first & third chromatography column is selected from Protein-A column, Hydrophobic interaction chromatography, cation exchange chromatography, and second chromatography column is anion exchange chromatography column

In an embodiment, the purification of CTLA4-Ig fusion protein by performing anion exchange chromatography, optionally further comprises one or more chromatography step can be employed before or after anion exchange chromatography.

In an embodiment, the one or more chromatography step selected from affinity chromatography, mixed mode chromatography, hydrophobic interaction chromatography and cation exchange chromatography can be performed before or after anion exchange chromatography.

In another embodiment, the invention is related to the purification of fusion protein by performing protein A chromatography, which is followed by anion exchange chromatography (AEX), optionally further comprises one chromatography step.

In another embodiment, the mixed mode chromatography and hydrophobic interaction chromatography can be utilised for removal of impurities selected from Host cell proteins (HCP), Host cell DNA (HCD), Pre-peak, Low molecular weight (LMW), aggregates and High molecular weight (HMW).

In an embodiment, the affinity chromatography column resin is selected from Protein A resin, Protein G resin, preferably Protein A resin. Protein A column chromatography resin is selected from Toyopearl AF-rProtein A HC-650, Mab Select Sure LX, MabSelect SuRe, MabSelectXtra, ProSep Ultra Plus, Eshmuno A.

In an embodiment, the first step of affinity chromatography comprises clarified harvest cell culture fluid (HCCF) that is obtained from suitable mammalian expression system. The pH of HCCF is adjusted to pH selected from about pH 8 to about pH 9, preferably pH 7±0.2 with 2 M Tris base just before loading onto the affinity column.

In an embodiment, prior to loading the Protein A column is equilibrated with a suitable buffer. In an embodiment, the suitable buffer is selected from Tris acetate, Tris-HCl buffer, Phosphate, Sodium Chloride, HEPES, Triethanolamine, Borate, Glycine-NaOH.

In preferred embodiment, the concentration of Tris-HCl is selected from about 5 mM to about 20 mM and Sodium chloride is selected from about 50 mM to about 200 mM at pH ranging from about pH 6.8 to about pH 7.5 and conductivity is selected from about from 10 mS/cm to about 25 mS/cm, preferably about 16 mS/cm.

In an embodiment, the concentration of buffer is 20 mM Tris HCl and 150 mM Sodium chloride, pH about 7.0±0.2 used to equilibrate the column with at least one column volumes, preferably for four column volumes. The flow rate can be selected from at about 50 cm/hr to at about 400 cm/hr, preferably 300 cm/hr.

In an embodiment, the loading of protein on column, the Protein A column can be washed one or multiple times by using the equilibrating buffer or by employing different buffers. The Protein A column is first washed with the equilibration buffer for at least 4-6 column volumes. This wash can optionally be followed by one or more wash. In another embodiment, the wash buffer is selected from urea, tween 80, isopropanol, NaCl, EDTA, Tris acetate, Tris-HCl, HEPES, Triethanolamine, Borate and Glycine-NaOH. The concentration of wash buffers is selected from 5 mM to about 200 mM and the pH of wash buffer is ranging from pH 6.8 to about pH 7.5.

In an embodiment, Protein A column comprises three wash buffers.
a) The first wash buffer comprises Tris HCl concentration selected from about 5 mM to about 20 mM and Nacl concentration selected from about 10 mM to about 150M Nacl, at pH 7.0±0.2.
b) The second wash buffer comprises Tris HCl concentration selected from about 5 mM to about 20 mM and NaCl concentration from about 1M to about 10 M at pH 7.0±0.2.
c) The third wash buffer comprises Tris HCl concentration selected from 5 mM to about 20 mM at pH 7.0±0.2

In an embodiment, the first wash buffer comprises 20 mM Tris HCl and 150 mM NaCl at pH 7.0±0.2.

In an embodiment, the second wash buffer comprises 20 mM Tris HCl and 1M NaCl at pH 7.0±0.2.

In an embodiment, the third wash buffer comprises 20 mM Tris HCl at pH 7.0±0.2.

In an embodiment, the Protein A column can then be eluted using an appropriate suitable buffer.

The linear gradient is achieved by using elution buffer selected from pH about 2 to 3.5. In an embodiment, the elution buffer comprises Tris Acetate concentration selected from about 100 mM to about 200 mM Tris Acetate, preferably 110 mM Tris Acetate in 2-3 column volume at pH 3.5, the conductivity is selected from about from 5 mS/cm to about 15 mS/cm, preferably about 12 mS/cm.

In an embodiment, the eluted fractions collected from ascending 10 mAU/cm to about descending 100 mAU/cm.

In an embodiment, the Protein A column further comprises neutralization wash with 20 mM Tris HCl at pH 7.0±0.2.

In an embodiment the protein mixture obtained from affinity chromatography column is subjected to suitable treatment to make the protein mixture suitable for loading onto AEX.

In another embodiment, the load preparation done at 1:1 dilution of Neutralized Protein A Elute (NPEL) (pH 7.5) with buffer, pH adjusted to 8.0±0.2 before loading.

In one embodiment, the anion exchange chromatography is selected from Poros XQ, Poros HQ DEAE, Sepharose fast flow, Fractogel® EMD DEAE (M), Toyopearl DEAE-650, Toyopearl DEAE-650, Nuvia Q.

In an embodiment, the anion exchange is strong anion exchange, preferably Poros XQ.

In an embodiment, the process does not include more than one strong anion exchange chromatography.

In an embodiment, the anion exchange chromatography is performed in the bind and elute mode. In another embodiment, the anion exchange chromatography is optionally performed in flow through mode. In an embodiment, the loading of the protein mixture to anion exchange chromatography column.

In an embodiment, the loading is performed at suitable pH selected from about 7.0 to about 7.5 and conductivity selected from about 5 mS/cm to about 8 mS/cm.

In an embodiment, pH of the anion exchange load is 7.2±0.2, conductivity of the sample is adjusted by diluting with WFI ratio selected from about 1:1, 1:1.1, 1:1.2, 1:1.3, 1:1.4, 1:1.5, more preferably 1:1.3.

In another embodiment, the conductivity of anion exchange load is adjusted with WFI from about 6.5 mS/cm.

In an embodiment, the anion exchange chromatography column is equilibrated with suitable buffer selected from histidine hydrochloride, tris acetate, sodium citrate, Sodium phosphate (NaP), citrate, Sodium chloride (NaCl) preferably Sodium phosphate (NaP) and Sodium chloride (NaCl) at pH selected from about 6 to about pH 9, preferably at pH 7.2±0.2, conductivity is selected from about 2.0 mS/cm to about 3.2 mS/cm, preferably 2.7±0.3 ms/cm.

In an embodiment, the concentration of equilibration buffer selected from about 5 mM to about 50 mM. In another embodiment, the concentration of selected from about 10 mM to about 50 mM. In another aspect of embodiment, the concentration is selected from about 15 mM to about 30 mM.

In an embodiment, the equilibration buffer comprises Sodium phosphate (NaP) at pH 7.5. In an embodiment, the equilibration buffer comprises 20 mM Sodium phosphate (NaP) at pH 7.2±0.2.

In an embodiment, the load preparation done with OP diluted with WFI in ratio 1:1.33 at pH 7.2±0.2. In a preferred embodiment, the protein mixture is loaded onto the AEX column. In another embodiment the flow rate can be selected from at about 50 cm/hr to at about 500 cm/hr, preferably 300 cm/hr.

In an embodiment, the wash buffer selected from Tris acetate, Tris HCl, Sodium citrate, Sodium chloride (NaCl), Sodium phosphate (NaP), at pH about 6.5 to about pH 7.5.

In an embodiment, the concentration of wash buffer selected from about 5 mM to about 30 mM. In another embodiment, the concentration of wash buffer selected from about 10 mM to about 30 mM. In another aspect of embodiment, the concentration of wash buffer selected from about 10 mM to about 20 mM.

In an embodiment, the suitable washing comprises:
(i) first wash with equilibration buffer at suitable pH 7.2±0.2 and/or conductivity 2.7±0.3 mS/cm;
(ii) second wash at the same pH as of the first wash buffer and/or a conductivity higher than the first wash buffer selected from about 5 mS/cm to about 20 mS/cm.

In an embodiment, the first wash buffer comprises about 20 mM to 30 mM Sodium phosphate (NaP) at pH 7.2±0.2.

In an embodiment, the second wash buffer comprises about 15 mM to about 30 mM Sodium phosphate (NaP) and less than 80 mM Sodium chloride (NaCl) preferably less than 60 mM NaCl at pH 7.2±0.2, and conductivity is selected from about 2.0 mS/cm to about 3.2 mS/cm, preferably 2.7±0.3 ms/cm.

In an embodiment, the second wash performed using step gradient selected from about 10%, 15%, 20%, 25%, 30% of buffer B, preferably 20% of buffer B in 5 CV.

In an embodiment, the elution buffer selected from Tris acetate, Acetate, Sodium citrate, Sodium chloride (NaCl), Sodium phosphate (NaP), at pH about 6.5 to about pH 7.5.

In an embodiment, the concentration of the high salt buffer used in elution buffer selected from about more than 100 mM. In an embodiment, the concentration of high salt buffer is selected from 100 mM to 1050 mM. In an embodiment, the concentration of high salt buffer is selected from 200 mM to 325 mM.

In an embodiment, the elution buffer comprises Sodium phosphate (NaP) concentration selected from 10 mM to about 50 mM. In an embodiment, the elution buffer comprises Sodium phosphate (NaP) concentrated selected from 10 mM, 20 mM, 30 mM, 40 mM, 50 mM, 60 mM, 70 mM, 80 mM, 90 mM and 100 mM.

In another embodiment, the elution buffer comprises Sodium chloride (NaCl) concentration selected from 0.1 M to about 1 M. In an embodiment, the elution buffer comprises Sodium chloride (NaCl) concentration selected from 0.1 mM, 0.2 mM, 0.3 mM, 0.4 mM, 0.5 mM, 0.6 mM, 0.7 mM, 0.8 mM, 0.9 mM, 1 mM, 1.2M and 1.5M.

In an embodiment, the elution is performed with an appropriate buffer. In another embodiment, the elution buffer can be one or mixture of more than one buffer.

In an embodiment, the protein is eluted by elution buffer comprising about 10 mM to 50 mM Sodium phosphate (NaP) and about 0.1 to 1 M Sodium chloride (NaCl), preferably 20 mM Sodium phosphate (NaP) and 0.3M Sodium chloride (NaCl) at pH 7.2±0.2, and conductivity is selected from about 20 mS/cm to about 50 mS/cm, preferably 30±3 mS/cm.

In an embodiment, the gradient was performed for column volume selected from 1CV, 2CV, 3CV, 4CV, 5CV, 6CV, 7CV, 8CV, 9CV, 10CV, 11CV, 12CV, 13CV, 14CV, 15CV, 16CV, 17CV, 18CV, 19CV and 20CV.

In an embodiment, the gradient of elution buffer is performed in anion exchange column for elution from about 20% to about 80% of buffer B, preferably about 20% to about 70% of 10 CV to 20 CV, preferably 15CV.

In certain embodiment, the elution is performed using step and/or linear gradient selected from about 1% to 10% step gradient and 10%, 15%, 20%, 25%, 30% of linear gradient of buffer B, preferably 20% of buffer B in 15 CV to 25 CV, more preferably in 15CV.

In an embodiment, the eluted fractions are collected from ascending 10 mAU/cm to about descending 80 mAU/cm in a fixed CV. In an embodiment, the eluted fractions collected from ascending 10 mAU/cm to about descending 100 mAU/cm.

The anion exchange reduces undesired glycan selected from high mannose & afucosylation.

In certain embodiment, the anion exchange chromatography reduces undesired glycan selected from high mannose and afucosylation by less than 50% preferably less than 70%.

In an embodiment, the CTLA4-Ig fusion protein has high mannose low or reduce by at least 50%.

In an embodiment, the CTLA4-Ig fusion protein has afucosylation reduce by at least 20%.

In an embodiment, the process improves more than 50% of high mannose in fusion protein.

In an embodiment, the high mannose is reduced about 50%, about 55%, about 60%, about 65%, about 70%, about 75%, about 80%.

In an embodiment, the fusion protein has afucosylation reduce about 20%, about 21%, about 22%, about 23%, about 24%, about 25%, about 26%, about 27%, about 28%, about 29%, and about 30%.

In an embodiment, the high mannose in fusion protein improved about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, and about 21%.

In an embodiment, the afucosylation in fusion protein improved about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, and about 21%.

In an embodiment, the galactosylation in fusion protein improved about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, and about 21%.

In an embodiment, the purified fusion protein comprises high mannose in range selected from about 0.05% to about 0.3%.

In an embodiment, the purified fusion protein comprises afucosylation in range selected from about 0.5% to about 3%.

In an embodiment, the purified fusion protein comprises galactosylation in range selected from about 25% to about 38%.

### Example 1—Purification of Fc-Fusion by Performing AEX (Anion Exchange Chromatography) Chromatography (50 L Scale)

Chromatographic processes were carried out using an AKTA Pure 150 system from Cytiva (formally known as GE Healthcare). Concentration of protein samples were determined by measuring absorbance at 280 nm using Shimadzu Spectrophotometer. AEX (Poros XQ) is obtained from Thermofisher.

Fc-fusion protein sample obtained from affinity chromatography loaded onto an anion exchange chromatography column, performing wash and eluting the Fc-fusion protein from the anion exchange chromatography column for which experiment design shown in table 1. The residence time is 4 min for all the phases.

The glycans were enzymatically removed with PNGase F treatment and labeled with fluorescence dye, glycans were separated and detected using Hydrophilic interaction liquid chromatography (HILIC) according to method available in the art and result shown in table 2.

TABLE 1
Experiment design for Anion exchange chromatography

| | | | Column |
|---|---|---|---|
| | | Residence | Volume |
| Step | Buffer | Time(min) | (CV) |
| Storage | WFI | 4 | 3 |
| buffer | | | |
| removal | | | |
| Sanitization | 0.5N Sodium Hydroxide | 4 | 3 |
| Charge | 20 mM Sodium Phosphate (NaP) + | 4 | 3 |
| | 1M Sodium chloride (NaCl), pH | | |
| | 7.2 ± 0.2 | | |
| Equilibration | 20 mM Sodium phosphate (NaP), | 4 | 10 |
| | pH 7.2 ± 0.2 | | |
| Load | OP is diluted with WFI in a ratio | 4 | Till |
| | of 1:1.33, pH 7.2 ± 0.2 | | loading |
| | | | volume |
| Wash 1 | 20 mM Sodium phosphate (NaP), | 4 | 10 |
| | pH 7.2 ± 0.2 | | |
| Wash 2 | 20 mM Sodium Phosphate (NaP) + | 4 | 20% B |
| | 60 mM Sodium chloride (NaCl), | | step |
| | pH 7.2 ± 0.2 | | gradient |
| | | | in 5 CV |
| Elution | 20 mM Sodium Phosphate (NaP) + | 4 | 20-70% B |
| | 300 mM Sodium chloride (NaCl), | | linear in |
| | pH 7.2 ± 0.2 | | 15 CV |
| Regeneration | 20 mM Sodium Phosphate (NaP) + | 4 | 5 |
| | 1M Sodium chloride (NaCl), pH | | |
| | 7.2 ± 0.2 | | |
| Sanitization | 0.5N Sodium Hydroxide | 4 | 3 |
| Storage | 0.1N Sodium Hydroxide | 4 | 3 |

TABLE 2
Quality Attributes for Anion exchange chromatography measured
by Hydrophilic interaction liquid chromatography (HILIC)

50 L Scale

| Quality Attributes | | NPEL | DS |
|---|---|---|---|
| N-Glycan | High Mannose | 0.81% | 0.19% |
| | Afucosylation | 1.15% | 0.87% |

1. A process of purifying a CTLA4-Ig fusion protein mixture, the purification process comprising:
a. loading the CTLA4-Ig fusion protein mixture onto anion exchange column with suitable buffer at suitable pH selected from pH 7.0 to 7.5;
b. optionally, the wash is performed;
c. eluting the CTLA4-Ig fusion protein mixture from anion exchange column;
wherein the CTLA4-Ig fusion protein mixture has low or reduce high mannose by at least 50%.

2. The process as claimed in claim 1, wherein the CTLA4-Ig fusion protein mixture has afucosylation reduce by at least 20%.

3. The process as claimed in claim 1, wherein the high mannose is reduced about 50%, about 55%, about 60%, about 65%, about 70%, about 75%, and about 80%.

4. The process as claimed in claim 1, wherein the fusion protein has afucosylation reduced about 20%, about 21%, about 22%, about 23%, about 24%, about 25%, about 26%, about 27%, about 28%, about 29%, and about 30%.

5. The process as claimed in claim 1, wherein process further comprises affinity chromatography, mixed mode chromatography, hydrophobic interaction chromatography and cation exchange chromatography performed before or after anion exchange chromatography.

6. The process as claimed in claim 5, wherein the anion exchange is selected from Poros XQ, Poros HQ DEAE, Sepharose fast flow, Fractogel® EMD DEAE (M), Toyopearl DEAE-650, Toyopearl DEAE-650, Nuvia Q.

7. The process as claimed in claim 6, wherein the Poros XQ anion exchange is strong anion exchange performs in bind elute mode comprising (i) loading the fusion protein mixture at suitable pH and/or conductivity for binding (ii) performing washing at suitable pH and/or conductivity (iii) eluting the fusion protein mixture at suitable pH and/or conductivity.

8. The process in as claimed in claim 7, wherein the loading of fusion protein mixture at suitable pH selected from about 7.0 to about 7.5 and conductivity selected from about 5 mS/cm to about 8 mS/cm.

9. The process as claimed in claim 8, wherein the suitable pH and/or conductivity is maintained with suitable buffer selected from sodium phosphate, Tris-HCl, Sodium citrate, and Sodium chloride.

10. The process as claimed in claim 1, wherein the suitable washing comprises:
(i) first wash with equilibration buffer at suitable pH 7.2±0.2 and/or conductivity 2.7±0.3 mS/cm;
(ii) second wash at the same pH as of the first wash buffer and/or a conductivity higher than the first wash buffer selected from about 5 mS/cm to about 20 mS/cm.

11. The process as claimed in claim 10, wherein the second wash buffer comprises sodium phosphate and sodium chloride, wherein the concentration of Sodium phosphate (NaP) selected from about 10 mM, about 20 mM, about 30 mM, about 40 mM, about 50 mM, about 60 mM, about 70 mM, about 80 mM, about 90 mM, and about 100 mM and Sodium chloride (NaCl) selected from about 10 mM, about 20 mM, about 30 mM, about 40 mM, about 50 mM, about 60 mM, about 70 mM, about 80 mM, about 90 mM, and about 100 mM.

12. The process as claimed in claim 1, wherein the elution is performed at suitable pH 7.2±0.2 and conductivity 30.0±3.0 mS/cm.

13. The process as claimed in claim 12, wherein the elution is performed with buffer sodium phosphate and sodium chloride.

14. The process as claimed in claim 13, wherein the elution is performed in linear gradient wherein the concentration of buffer increased from about 20% to about 80%, preferably about 20% to about 70% for column volume selected from about 1CV, about 2CV, about 3CV, about 4CV, about 5CV, about 6CV, about 7CV, about 8CV, about 9CV, about 10CV, about 11CV, about 12CV, about 13CV, about 14CV, about 15CV, about 16CV, about 17CV, about 18CV, about 19CV and about 20CV.

15. The process as claimed in claim 14, wherein the elution starts, and fractions collected at an ascending value of about 10 mAU/cm and descending value of about 80 mAU/cm.

16. The process as claimed in claim 1, the anion exchange chromatography performed in bind and elute mode.

17. The process as claimed in claim 9, wherein the concentration of suitable buffer selected from about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1M, about 1.2M, and about 1.5M.

18. The process as claimed in claim 10, wherein the equilibration buffer comprises Sodium phosphate (NaP), the concentration selected from about 10 mM, about 20 mM, about 30 mM, about 40 mM, about 50 mM.

19. The process as claimed in claim 13, wherein the elution buffer comprises Sodium phosphate (NaP) concentration selected from about 10 mM, about 20 mM, about 30 mM, about 40 mM, about 50 mM, about 60 mM, about 70 mM, about 80 mM, about 90 mM, and about 100 mM and sodium chloride (NaCl) concentration selected from about 0.1 mM, about 0.2 mM, about 0.3 mM, about 0.4 mM, about 0.5 mM, about 0.6 mM, about 0.7 mM, about 0.8 mM, about 0.9 mM, about 1M, about 1.2M, and about 1.5M.

20. A pharmaceutical composition of CTLA4-Ig fusion protein comprises high mannose in range selected from about 0.05% to about 0.3%, afucosylation in range selected from about 0.5% to about 3%, and galactosylation in range selected from about 25% to about 38% detected by Hydrophilic interaction liquid chromatography (HILIC).
